(19) United States
(12) Patent Application Publication
(10) Pub. No.: US 2025/0345095 A1
(43) Pub. Date: Nov. 13, 2025

(54) DISTRACTOR DEVICE

(71) Applicant: Karl Leibinger Asset Management GmbH & Co. KG, Mühlheim an der Donau (DE)

(72) Inventor: Christoph URBANKE, Donaueschingen (DE)

(21) Appl. No.: 19/200,033

(22) Filed: May 6, 2025

(30) Foreign Application Priority Data
May 7, 2024 (DE) 102024112763.5

(51) Int. Cl. A61B 17/64

(52) U.S. Cl. CPC A61B 17/6433

(57) ABSTRACT

A distractor device includes a first and a second bone plate, a housing fixedly connected to the first bone plate, and two lateral faces that extend in parallel with each other and are at least partially curved. A slide is fixedly connected to the second bone plate and is arranged so as to be movable along the lateral faces such that a non-linear movement path of the slide is defined by the curvature of the lateral faces. A main shaft has an external thread. A guide body is operatively connected to the slide and has an internal thread in engagement with the external thread of the main shaft such that rotation of the main shaft effects a displacement of the guide body along the main shaft and thus a displacement of the slide along the non-linear movement path. The main shaft is located within a receiving space in the housing.

### CROSS-REFERENCE TO RELATED APPLICATION

The present application is related and has right of priority to German Patent Application No. DE102024112763.5 filed on May 7, 2024, which is incorporated by reference in its entirety for all purposes.

### TECHNICAL FIELD

The present invention relates generally to a distractor device for implantation in a patient.

### BACKGROUND

Distractors are used in the field of medicine in particular to pull bone fragments apart by applying external mechanical stress. The prior art describes, in particular, linear distractors which effect a linear displacement, as well as non-linear, or curvilinear, distractors which effect a non-linear displacement along a curved path such as along a circular segment.

Furthermore, it is known to effect a distraction along a curved path, for example, in a treatment on a human jawbone by using two linear distractors.

CN 104 586 481 A describes a curved distractor which includes a threaded rod and a drive, which are interconnected via a universal joint. Rotating the threaded rod about the longitudinal axis of the threaded rod effects a displacement of a slider, which is guided by a curved guide rail. The slider and the guide rail are each connected to a bone plate. Rotating the threaded rod effects a curvilinear distraction between the bone plates.

The threaded rod described in CN 104 586 481 A undergoes a pivoting motion during the distraction, and therefore surrounding tissue can be damaged.

### BRIEF SUMMARY

Example aspects of the invention provide a non-linear distractor with which surrounding tissue is not damaged by the threaded rod.

In example embodiments of the invention, a distractor device for implantation in a patient enables a non-linear, or curvilinear, distraction of bone to be effected, wherein the threaded rod used for the distraction is enclosed in a housing, enabling surrounding tissue to be protected from the threaded rod. Such a curvilinear distraction is needed, for example, in treatments on a human jawbone.

The distractor device according to example aspects of the invention includes a first bone plate and a second bone plate. The bone plates are used to attach the distractor device to bones, or parts of bones, of a patient. By actuating the distractor device, the second bone plate can be displaced relative to the first bone plate.

The first bone plate is fixedly connected to a housing. The housing has a first lateral face and a second lateral face, which extend in parallel with one another and are at least partially curved. The lateral faces are substantially lateral outer surfaces of the housing and are used to guide a slide. The curvature can be defined depending on the requirement of the desired therapy.

The slide guided by the lateral faces is fixedly connected to the second bone plate and arranged so as to be movable along the lateral faces of the housing, so that a non-linear movement path of the slide is defined by the curvature of the lateral faces. The curvature of the housing therefore effects the curved (curvilinear) distraction of the second bone plate relative to the first bone plate.

The distractor device also includes a main shaft. The main shaft has an external thread. The main shaft therefore has a portion which is formed as a threaded rod. The rotation of the main shaft takes place about its longitudinal axis.

The slide is operatively connected to a guide body, which has an internal thread. The internal thread is in engagement with the external thread of the main shaft, so that a rotation of the main shaft effects a displacement of the guide body along the main shaft. Due to the operative connection between the guide body and the slide, a displacement of the guide body effects a displacement of the slide along the non-linear movement path defined by the lateral faces.

The main shaft is located within a receiving space in the housing. In this way, the surrounding tissue is protected from the main shaft.

Preferably, the main shaft is located completely within the receiving space in the housing, so that neither end of the main shaft protrudes from the housing. As a result, particularly good protection of the surrounding tissue is achieved.

Preferably, the receiving space in the housing is defined by the inner faces of the first and the second lateral faces and by a top face, which connects the first and the second lateral faces to one another. In other words, the receiving space is enclosed on three sides. As a result, in addition to the protection of the surrounding tissue, a simple mounting of the distractor device is achieved.

Preferably, the slide is guided by the outer faces of the lateral faces. This allows for a simple construction of the distractor device.

Preferably, the distractor device includes a drive shaft, which is connected to the main shaft via a joint. By the joint, a rotation of the drive shaft is transmitted onto the main shaft. One end of the drive shaft protrudes from the housing. Due to the joint, an angle can be achieved between the drive shaft and the main shaft. As a result, when the distractor device is implanted, a constant position of the drive shaft is made possible.

The first bone plate and the second bone plate preferably each include multiple through-holes for attaching the bone plates to a patient's bone.

The main shaft can preferably be mounted in the housing at a second end, which is opposite the first end. For this purpose, a corresponding bore, or receptacle, can be formed in the housing. The main shaft is preferably mounted slidably in the housing. Particularly preferably, one end of the main shaft has a tapered projection, which is mounted in a recess, or a hole, at one end of the housing (in the longitudinal direction). The projection extends preferably coaxially to the external thread in order to effect a uniform rotation of the main shaft and thus a uniform distraction.

The pitch of the thread can preferably be selected such that an even number of complete turns of the main shaft, or of the drive shaft, effects a specified distraction. Thus, for example, the patient or the treating physician can effect a specified number of turns once each day or multiple times each day in order to achieve the daily distraction desired for the therapy.

Furthermore, the distractor device can include a drive head, which is operatively connected to the drive shaft via an anti-relapse mechanism and preferably includes an interface for an external tool for rotating the drive shaft. Via the interface, the desired daily distraction, or the daily distraction prescribed by the physician, can be effected. Rotating the drive head by the external tool effects a rotation of the drive shaft. The joint transmits the rotation of the drive shaft onto the main shaft. The rotation of the main shaft effects a displacement of the guide body, which in turn entrains the slide and the second bone plate and displaces these relative to the first bone plate.

The anti-relapse mechanism prevents the patient, or the physician, from rotating the drive head in the wrong direction. Thus, it can be ensured that, when the drive head is rotated, only a positive distraction can ever be effected and a negative distraction can never be effected. Positive distraction means, in this case, an increase in the distance between the two bone plates. A negative distraction correspondingly means a decrease in the distance between the bone plates.

The anti-relapse mechanism preferably includes multiple teeth, which are formed on the drive head and each have a flat flank and a steep flank. Furthermore, the anti-relapse mechanism preferably includes a pawl mounted in the housing, which can slide over the flat flanks and lock against the steep flanks. The teeth are oriented in accordance with the pitch of the thread such that only a positive distraction can be effected. The teeth are preferably arranged around the circumference of the drive head.

Furthermore, a spring is preferably located in the housing, which generates a preloading of the pawl against the teeth. The spring can preferably generate a compressive force of the pawl against the teeth. In alternative example embodiments, the spring can also be designed to generate a tensile force and can be correspondingly arranged.

During rotation in the intended direction, the pawl slides over a flat flank. The flat flank can preferably deflect the pawl counter to the spring force. At the end of the flat flank, such as at the crown of the tooth, the pawl then jumps outward by the height of the steep flank in the direction of the spring force. Rotation counter to the intended direction is prevented by the steep flank since the pawl then impacts a steep flank.

The anti-relapse mechanism can preferably include an adjusting pin. The adjusting pin is used to lock the pawl in a position in which the pawl cannot interact with the teeth. Thus, the anti-relapse mechanism can be deactivated and rotation in the opposite direction can be permitted. The adjusting pin can preferably be locked in the open position similarly to a bayonet connection.

The joint is preferably a ball joint. Such a joint has the advantage that, unlike a universal joint, the joint does not require a bolt. It can therefore be particularly easily assembled.

The ball joint includes, according to a preferred example embodiment, a ball having two grooves which are perpendicular to one another and extend around the circumference. The grooves act as sliding bearings for receptacles on the shafts.

Preferably, a receptacle is formed at one end of the main shaft, which receptacle is mounted slidably in a first groove in the ball. Correspondingly, a receptacle is preferably formed at one end of the drive shaft, which receptacle is mounted slidably in a second groove in the ball. The receptacles preferably have a semicircular, or U-shaped, cross-section, the internal radius of which corresponds to the circumference of the grooves in the ball. The thickness of the receptacles corresponds to the width of the grooves, so that a precisely-fitting sliding joint can be provided.

The guide body is preferably mounted so as to be displaceable perpendicularly to the longitudinal axis of the main shaft. In other words, the guide body can be displaced in order to follow the curvature of the lateral faces, so that the intended curvilinear distraction can be carried out. A connecting plate, which is fixedly connected to the second bone plate or to the slide, can be provided for support. The connecting plate can also facilitate the mounting of the distractor device. A recess is located in the connecting plate preferably perpendicularly to the longitudinal axis of the main shaft, in which recess the guide body can move perpendicularly to the longitudinal axis of the main shaft during the displacement of the slide.

### BRIEF DESCRIPTION OF THE DRAWINGS

Exemplary embodiments of the invention are described in detail on the basis of the figures. Wherein:

FIG. 1 shows a distractor device according to one exemplary embodiment when viewed from below;

FIG. 2 shows the example distractor device when viewed from above;

FIG. 3a and FIG. 3b show side views of the example distractor device;

FIG. 4 shows a sectional view in parallel with the view from above;

FIG. 5 shows a lateral sectional view in parallel with the axis of rotation;

FIG. 6 shows a perspective view of a region of the example distractor device;

FIG. 7a and FIG. 7b show detailed views of a ball joint of the example distractor device;

FIG. 8a and FIG. 8b show detailed views of a drive shaft of the example distractor device;

FIG. 9a and FIG. 9b show detailed views of a ball of the ball joint of the example distractor device; and

FIG. 10a and FIG. 10b show detailed views of a main shaft of the example distractor device.

The accompanying figures are intended to provide a further understanding of the example embodiments of the invention. The figures illustrate embodiments and, in the context of the description, serve to explain the principles and concepts of the invention. Other example embodiments and many of the aforementioned advantages can be gathered from the drawings. The elements of the drawings are not necessarily drawn to scale in relation to each other.

In the figures of the drawing, the same, functionally identical and similarly acting elements, features and components are each provided with the same reference signs, unless otherwise stated.

### DETAILED DESCRIPTION

Reference will now be made to embodiments of the invention, one or more examples of which are shown in the drawings. Each embodiment is provided by way of explanation of the invention, and not as a limitation of the invention. For example, features illustrated or described as part of one embodiment can be combined with another embodiment to yield still another embodiment. It is intended that the present invention include these and other modifications and variations to the embodiments described herein.

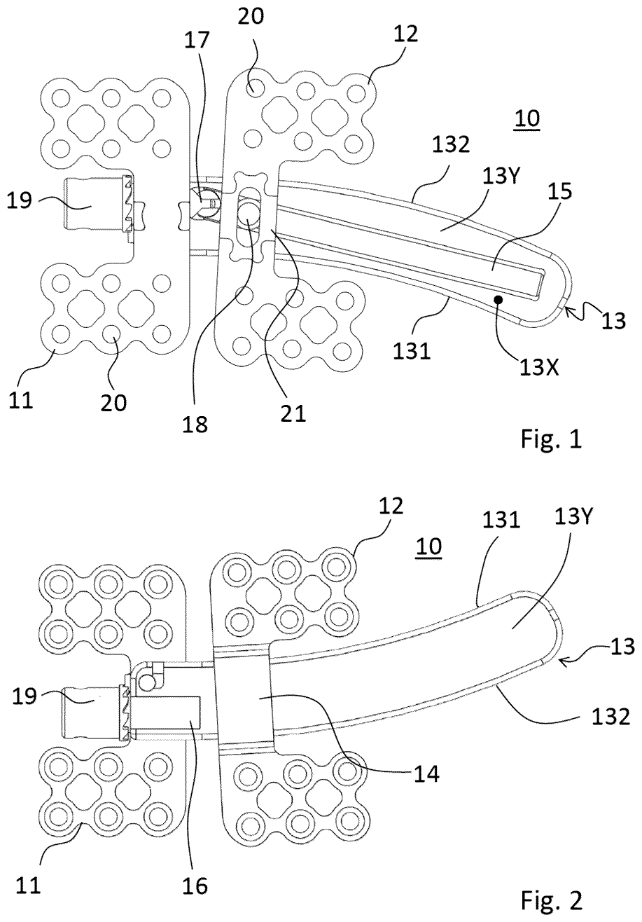

FIG. 1 shows a distractor device 10 according to one exemplary embodiment of the invention when viewed from below. The distractor device 10 includes a housing 13, which is curved along a circular arc, is made of a metal alloy suitable for implants, and is solid. A receiving space 13X for a main shaft 15 is formed in the housing 13, so that the main shaft 15 can be inserted into the housing 13 from below.

The housing has a first lateral face 131 and a second lateral face 132 extending perpendicularly to the image plane. The lateral faces 131 and 132 are parallel with one another and each have a first radius of curvature and a second radius of curvature, which is greater than the first radius of curvature. The outer surfaces of the lateral faces 131, 132 serve as sliding surfaces for a slide 14 and guide the slide 14 (see FIG. 2) along the housing 13. The curvature and size of the housing 13 and of the lateral faces 131, 132 of the housing can be selected according to the required therapy. Thus, the distractor device 10 can be adapted, or produced, precisely for the specified purpose of use. The two lateral faces 131, 132 are interconnected by a top face 13Y. The receiving space 13X is thus defined by the inside of the lateral faces 131, 132 and by the inside of the top face 13Y. The housing 13 therefore has a U-shaped cross-section in the guide area of the slide 14.

A first bone plate 11 is fixedly connected to the housing 13. For this purpose, the first bone plate 11 can be, for example, screwed together with, welded to, or press fit with the housing 13. Furthermore, the housing 13 and the first bone plate 11 can be produced as a single piece. The first bone plate 11 is substantially flat in a plane perpendicular to the lateral faces 131, 132. A second bone plate 12 has a shape similar to that of the first bone plate 11.

In order to attach the distractor device 10 to the patient's bone, the first and the second bone plates 11, 12 each include multiple holes. Furthermore, larger cut-outs can be provided in the bone plates 11, 12 in order to reduce the weight of the bone plates 11, 12 and thus the amount of foreign material introduced into the patient.

The main shaft 15 has an external thread. A guide body 18 having an internal thread is in engagement with the external thread of the main shaft 15. Rotation of the main shaft 15 about the longitudinal axis of the main shaft 15 effects a corresponding displacement of the guide body 18, from left to right in FIG. 1. An extension of the guide body 18 engages into a recess in a connecting plate 21. Via the extension, the guide body 18 entrains the connecting plate 21 during the displacement. The connecting plate 21 is fixedly connected to the second bone plate 12. Thus, rotation of the main shaft 15 effects a displacement of the second bone plate 12 away from the first bone plate 11.

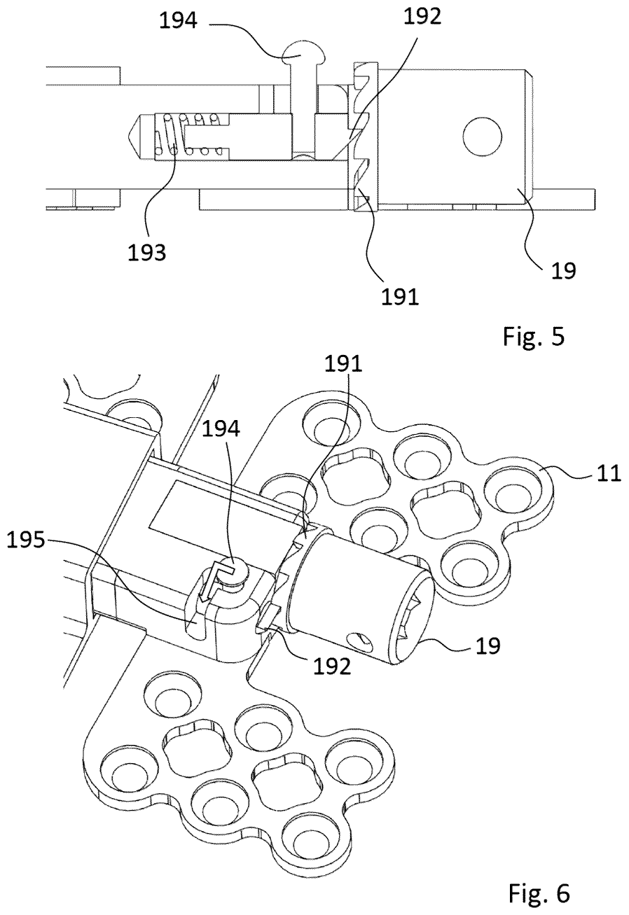

The main shaft 15 is connected to a drive shaft 16 (see FIG. 2) via a ball joint 17. The drive shaft 16 is connected via an anti-relapse mechanism (see FIGS. 5 and 6) to a drive head 19, which acts as an interface for an external tool for rotating the drive shaft 16. The drive head 19 can be connected to the drive shaft 16, or the drive head 19 and the drive shaft 16 can be produced as a single piece.

FIG. 2 shows the distractor device 10 when viewed from above. The slide 14, which is fixedly connected to the second bone plate 12, can be seen in this representation. The slide 14 is guided by the lateral faces 131 and 132 of the housing 13. The surfaces of the corresponding inner lateral faces of the slide 14 each have radii of curvature corresponding to the lateral faces 131 and 132 in order to allow the slide to smoothly glide on the housing 13 without tilting.

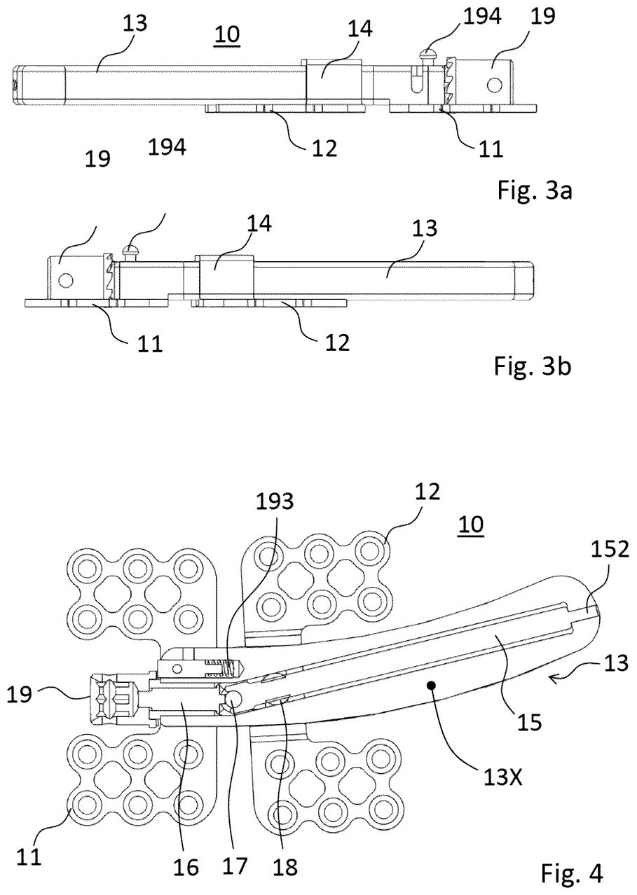

FIGS. 3a and 3b each show side views of the distractor device shown in FIG. 1.

FIG. 4 shows a sectional view in parallel with the view from above shown in FIG. 2. In this view, it can be seen how a second end 152 of the main shaft 15 is mounted slidably in a bore at the end of the housing 13. Furthermore, the guide body 18 is apparent, the internal thread of which is seated on the external thread of the main shaft 15.

FIG. 5 shows a side sectional view through an adjusting pin 194 of an anti-relapse mechanism of the distractor device 10. Additional details of the anti-relapse mechanism are shown in the detailed view in FIG. 6. FIG. 6 shows a perspective view of one region of the distractor device 10.

The drive head 19 has multiple circumferentially arranged teeth 191, which interact with a pawl 192. The pawl 192 is pressed against the teeth 191 by a spring 193. The teeth 191 each have a flat flank and a steep flank. When the drive head 19 rotates in the clockwise direction, the pawl 192 slides over each of the flat flanks. When there is rotation in the counterclockwise direction, the pawl 192 blocks since the pawl 192 then impacts the steep flanks.

The anti-relapse mechanism can be released by moving the adjusting pin 194. As is apparent from FIGS. 5 and 6, the adjusting pin 194 can be moved toward the left counter to the spring force of the spring 193, whereby the pawl 192 releases the teeth 191. Similar to a bayonet coupling, the adjusting pin 194 can be locked against the spring 193 in a deviated position by swiveling the adjusting pin 194 into the recess 195 shown in FIG. 6. This procedure is illustrated by the angled arrow.

The anti-relapse mechanism can prevent the main shaft 15 from being rotated in the wrong direction. In other words, the anti-relapse mechanism can prevent the distance between the bone plates 11, 12 from decreasing during the therapy. This is important, in particular, when the patient is to operate the distractor device 10 themself.

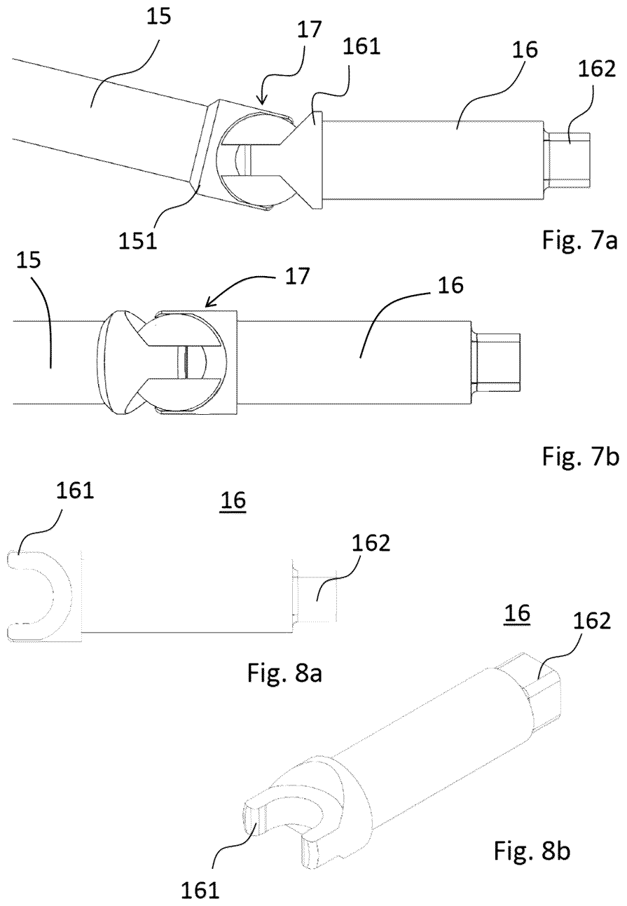

FIGS. 7a, 7b, 8a, 8b, 9a, 9b 10a, and 10b show detailed views of the ball joint 17 of the distractor device 10, of the drive shaft 16, of a ball 171 of the ball joint 17, and of the main shaft 15.

The ball joint 17 connects a first end 151 of the main shaft 15 to a first end 161 of the drive shaft 16. The main shaft 15 has an opposite, second end 152, which is used to mount the main shaft 15 in the housing 13. The drive shaft 16 has a second end 162, which is opposite the first end 161 and is coupled to the drive head 19. As is shown in FIGS. 7a, 7b, and FIGS. 8a, 8b, the second end 162 can have a square shape. The drive head 19 can have a corresponding square socket, which can accommodate the second end 162 of the drive shaft 16 in order to establish a force-locking connection. The drive shaft 16 is mounted slidably in the housing 13 between the first end 161 and the second end 162 in a corresponding longitudinal bore having a circular cross-section.

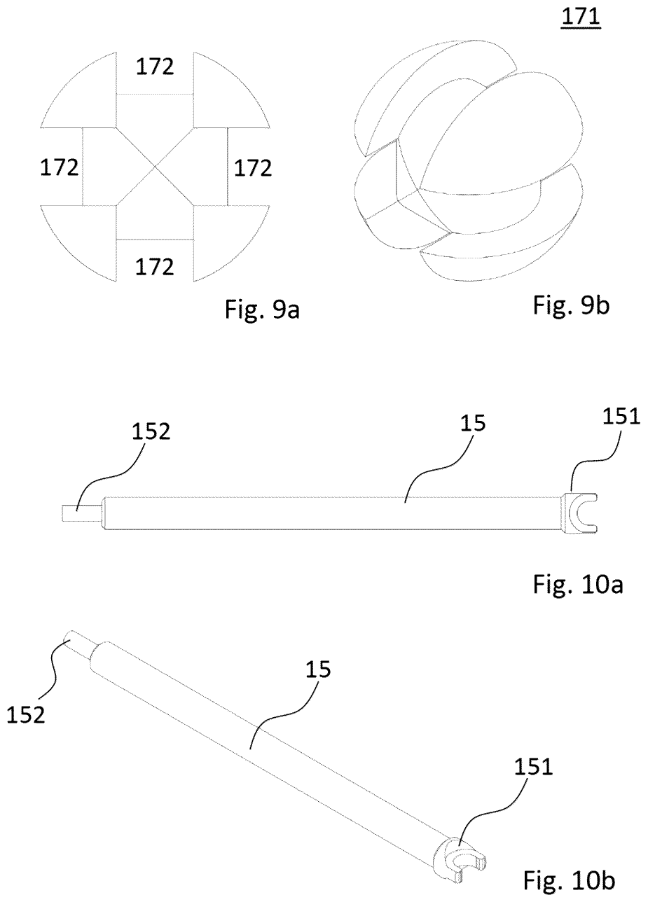

The ball 171 of the ball joint 17 includes, as shown in FIGS. 9a and 9b, two grooves 172 which are perpendicular to one another and extend around the circumference. Receptacles formed on the first shaft ends 151 and 161 engage around the ball 171 at the grooves 172. In this way, the ball joint 17 can be easily assembled, as a result of which the mounting of the distractor device 10 is simplified. The ball joint 17 also enables the rotation input via the drive shaft 16 to be particularly precisely transmitted onto the main shaft 15.

The receptacles on the first shaft ends 151, 161 can preferably be designed such that the ball 171 can be clipped in and then retained by the receptacles. For this purpose, the limbs of the substantially U-shaped receptacles can have an elasticity necessary for this purpose. For this purpose, the thickness, or strength, of the limbs can be correspondingly adapted.

All components of the distractor device 10 are preferably produced from a metal alloy which is particularly suitable for implants and, particularly preferably, can contain titanium as a main component. For example, the individual components can be produced by a material-removing method or by an additive method, or by a combination of these methods.

The distractor device 10 can effect a non-linear distraction without the need to pivot the main shaft 15. Furthermore, by using the ball joint 17, a particularly easy and time-saving production of the distractor device can be achieved. Due to the fact that a straight and rigid main shaft 15 can be used, which is located and mounted in the housing 13 in a protected manner, a particularly accurate and easily controllable displacement of the bone plates 11, 12 counter to one another can be effected. The slide 14 slides on the curved surfaces of the lateral faces 131, 132; a trajectory having any type of curvature can be defined by the shape of the lateral faces.

In the present description, several features have been labeled with the terms “first” and “second.” These designations only serve to clearly distinguish the individual features. In particular, no spatial or functional arrangement or prioritization should be inferred from this.

If a list of alternatives is followed by “or” in this application, it should be understood that both the listed alternatives by themselves and, if appropriate, a combination of several or all listed alternatives are to be understood.

Modifications and variations can be made to the embodiments illustrated or described herein without departing from the scope and spirit of the invention as set forth in the appended claims. In the claims, reference characters corresponding to elements recited in the detailed description and the drawings may be recited. Such reference characters are enclosed within parentheses and are provided as an aid for reference to example embodiments described in the detailed description and the drawings. Such reference characters are provided for convenience only and have no effect on the scope of the claims. In particular, such reference characters are not intended to limit the claims to the particular example embodiments described in the detailed description and the drawings.

1-13. (canceled)

14. A distractor device (10) for implantation in a patient, comprising:
a first bone plate (11) and a second bone plate (12);
a housing (13) fixedly connected to the first bone plate (11), the housing (13) comprising a first lateral face (131) and a second lateral face (132) that extend in parallel with each other and are at least partially curved;
a slide (14) fixedly connected to the second bone plate (12) and arranged so as to be movable along the first and second lateral faces (131, 132) of the housing (13) such that a non-linear movement path of the slide (14) is defined by curvature of the lateral faces (131, 132);
a main shaft (15) comprising an external thread; and
a guide body (18) operatively connected to the slide (14), the guide body (18) comprising an internal thread in engagement with the external thread of the main shaft (15) such that rotation of the main shaft (15) effects displacement of the guide body (18) along the main shaft (15) and thus displacement of the slide (14) along the non-linear movement path,
wherein the main shaft (15) is located at least partially within a receiving space (13X) in the housing (13).

15. The distractor device (10) of claim 14, wherein the main shaft (15) is located completely within the receiving space (13X) in the housing (13).

16. The distractor device (10) of claim 14, wherein the receiving space (13X) is defined by inner surfaces of the first and the second lateral faces (131, 132) and by a top surface (13Y) that connects the first and the second lateral faces (131, 132).

17. The distractor device (10) of claim 14, wherein the slide (14) is guided by outer surfaces of the lateral faces (131, 132).

18. The distractor device (10) of claim 14, further comprising a drive shaft (16) connected to the main shaft (15) via a joint (17) for transmitting rotation of the drive shaft (16) onto the main shaft (15), wherein one end of the drive shaft (16) protrudes from the housing (13).

19. The distractor device (10) of claim 18, wherein the main shaft (15) is mounted in the housing (13) at an end (152) of the housing (13) opposite the joint (17).

20. The distractor device (10) of claim 18, wherein the joint (17) comprises a ball joint.

21. The distractor device (10) of claim 20, wherein the ball joint comprises a ball (171) with grooves (172) perpendicular to one another and extending around a circumference of the ball (171).

22. The distractor device (10) of claim 21, wherein:
a first receptacle is formed at one end (151) of the main shaft (15);
the one end (151) of the main shaft (15) mounted slidably in a first groove (172) in the ball (171);
a second receptacle is formed at one end (161) of the drive shaft (16); and
one end (161) of the drive shaft (16) mounted slidably in a second groove (172) in the ball (171).

23. The distractor device (10) of claim 18, further comprising a drive head (16) operatively connected to the drive shaft (16) via an anti-relapse mechanism, the drive head (16) comprising an interface for an external tool for rotating the drive shaft (16).

24. The distractor device (10) of claim 23, wherein the anti-relapse mechanism comprises:
a plurality of teeth (191) formed on the drive head (19), each of the teeth (191) comprising a flat flank and a steep flank;
a pawl (192) mounted in the housing (13), the pawl (192) slidable over the flat flanks and lockable against the steep flanks; and
a spring (193) located in the housing (13) and configured for generating a preloading of the pawl (192) against the teeth (191).

25. The distractor device (10) of claim 24, wherein:
the anti-relapse mechanism further comprises an adjusting pin (194); and
the adjusting pin (194) is configured to lock the pawl (192) in a position in which the pawl (192) cannot interact with the teeth (191).

26. The distractor device (10) of claim 14, wherein the guide body (18) is supported at a connecting plate (21) so as to be displaceable perpendicularly to a longitudinal axis of the main shaft (15), the connecting plate (21) fixedly connected to the second bone plate (12) or to the slide (14).
